(19) United States
(12) Patent Application Publication
(10) Pub. No.: US 2020/0345606 A1
(43) Pub. Date: Nov. 5, 2020

(54) COMPOSITIONS AND METHODS FOR REMOVING SEMI-PERMANENT HAIR DYES

(71) Applicant: L'Oreal, Paris (FR)

(72) Inventors: Guojin ZHANG, Westfield, NJ (US); Gregory PLOS, Paris (FR)

(21) Appl. No.: 16/399,353

(22) Filed: Apr. 30, 2019

(51) Int. Cl. A61K 8/46; A61K 8/34; A61K 8/36; A61K 8/86; A61K 8/49; A61Q 5/02

(52) U.S. Cl. CPC A61K 8/463; A61K 8/34; A61Q 5/02; A61K 8/86; A61K 8/4973; A61K 8/36

(57) ABSTRACT

The disclosure relates to compositions and methods for removing semi-permanent hair dyes from hair. The compositions according to the disclosure comprise one or more anionic surfactants, one or more co-surfactants, and optionally one or more cyclic compounds. The methods comprise applying the compositions according to the disclosure to the hair.

### TECHNICAL FIELD

The present disclosure relates to compositions and methods for removing semi-permanent hair color from hair fibers.

### BACKGROUND

It is known to color hair with a permanent or semi-permanent hair color composition. Permanent hair color compositions contain oxidation dye precursors which are colorless or weakly colored compounds which, in combination with oxidizing products, can give rise to colored compounds by an oxidative condensation process. Permanent hair color compositions impart color to the hair that does not wash out.

In contrast, semi-permanent hair color compositions alter the color of the hair by depositing colored chemicals directly onto the surface of the hair. These so-called “direct dyes” are nonionic or ionic dyes capable of producing a more or less pronounced change of the natural color of the hair. Semi-permanent hair dyes will gradually wash out over time, generally in about 6-12 washings, although depending on the particular direct dye in the composition, the hair color can last longer as some direct dyes are more difficult to remove from the hair fibers than others. Therefore, consumers may choose a semi-permanent hair color composition when it is desired to only temporarily alter the color of the hair. However, there is a need for compositions and methods for removing semi-permanent hair color in less time than the 6-12 or more washings that most semi-permanent hair dyes generally require to wash out, as consumers may prefer to change the color of their hair without having to wait for the direct dyes to wash out.

### SUMMARY

The present disclosure relates to compositions and methods for removing semi-permanent hair dyes from hair. The compositions according to the disclosure comprise one or more anionic surfactants, one or more co-surfactants, and optionally one or more cyclic compounds. The methods comprise applying the compositions according to the disclosure to the hair.

According to one embodiment, the disclosure relates to compositions comprising at least one anionic surfactant and at least one co-surfactant to the hair fibers, which may be useful for removing semi-permanent hair dyes from hair fibers.

In a further embodiment, the compositions comprise at least one anionic surfactant chosen from sulfate anionic surfactants, sulfonate anionic surfactants, carboxylate anionic surfactants, or salts thereof; at least one co-surfactant chosen from C1-C5 alcohols or C1-C5 carboxylic acids; and optionally at least one cyclic compound chosen from aromatic alcohols or cyclic carbonates.

In yet further embodiments, the compositions comprise at least one anionic surfactant chosen from sodium laureth sulfate, present in an amount ranging from about 1% to about 25% by weight, relative to the total weight of the composition; at least one co-surfactant chosen from C1-C5 alcohols or C1-C5 carboxylic acids, present in an amount ranging from about 5% to about 25% by weight, relative to the total weight of the composition; and at least one cyclic compound chosen from aromatic alcohols or cyclic carbonates, present in an amount ranging from about 1% to about 25% by weight, relative to the total weight of the composition.

In still further embodiments, the disclosure relates to methods of removing direct dyes from hair fibers, the methods comprising: applying a composition comprising at least one anionic surfactant, at least one co-surfactant, and optionally at least one cyclic compound to the hair fibers, and rinsing the composition from the hair fibers, wherein the at least one co-surfactant is chosen from C1-C5 alcohols or C1-C5 carboxylic acids.

### DESCRIPTION

The present disclosure relates to compositions and methods for removing semi-permanent hair color from hair fibers. The compositions comprise at least one anionic surfactant, at least one co-surfactant, and optionally at least one cyclic compound. The methods comprise applying the compositions to the hair in order to remove semi-permanent hair dye from the hair fibers.

### Anionic Surfactants

The compositions according to the disclosure comprise at least one anionic surfactant. The term “anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from, for example, —CO₂H, —CO₂⁻, —SO₃H, —SO₃⁻, —OSO₃H, —OSO₃⁻, —H₂PO₃, —HPO₃⁻, —PO₃²⁻, —H₂PO₂⁻, ═HPO₂, —HPO₂⁻, ═P₂⁻, ═POH, and ═PO⁻ groups.

The anionic surfactants may be, for example, sulfate, sulfonate, carboxylic (or carboxylate) surfactants, or mixtures thereof. The sulfate, sulfonate, or carboxylic (or carboxylate) surfactants may, in various embodiments, comprise saturated or unsaturated hydrocarbon chains. The anionic surfactants may optionally be in salt form, or in the form of alkali metal or alkaline-earth metal, ammonium, or amino alcohol salts.

Sulfate anionic surfactants comprise at least one sulfate function. The sulfate anionic surfactants that may be used comprise at least one sulfate function (—OSO₃H or —OSO₃). They may be chosen from, by way of non-limiting example, alkyl or alkenyl sulfates, alkyl or alkenyl ether sulfates, alkylamido or alkenylamido ether sulfates, alkylaryl or alkenylaryl polyether sulfates, monoglyceride sulfates, and salts of these compounds. In various embodiments, the alkyl or alkenyl groups of these compounds comprise up to 30 carbon atoms, such as, for example from 6 to 30 carbon atoms, such as from 8 to 28, from 8 to 22 or from 8 to 18 carbon atoms, and the aryl group may optionally denote a phenyl or benzyl group. In at least some embodiments, these compounds may optionally be polyoxyalkylenated, especially polyoxyethylenated, for example comprising from 1 to 50 ethylene oxide units, such as from 2 to 10 ethylene oxide units.

In certain embodiments, sulfate anionic surfactants are chosen from alkyl or alkenyl sulfates, such as C6-C24 alkyl or alkenyl sulfates or C12-C20 alkyl or alkenyl sulfates, or from alkyl or alkenyl ether sulfates, optionally having from 2 to 20 ethylene oxide units, such as C6-C24 alkyl or alkenyl ether sulfates, or C12-C20 alkyl or alkenyl ether sulfates.

Sulfonate anionic surfactants comprise at least one sulfonate function (—SO₃H or —SO₃⁻) and may optionally also comprise one or more sulfate functions.

The sulfonate anionic surfactants that may be used comprise at least one sulfonate function (—SO₃H or —SO₃⁻). They may be chosen from the following compounds: alkylsulfonates, alkenylsulfonates, alkylamidesulfonates, alkenylamidesulfonates, alkylarylsulfonates, alkenylarylsulfonates, α-olefinsulfonates, paraffin sulfonates, alkylsulfosuccinates, alkenylsulfosuccinates, alkyl or alkenyl ether sulfosuccinates, alkylamidesulfosuccinates, alkenylamidesulfosuccinates, alkylsulfoacetates, alkenylsulfoacetates, N-acyltaurates, acylisethionates, alkylsulfolaurates, alkenylsulfolaurates, and salts of these compounds; the alkyl or alkenyl groups of these compounds comprising up to 30 carbon atoms, such as, for example, from 6 to 30 carbon atoms, such as from 8 to 28, from 8 to 22 or from 8 to 18 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

In certain embodiments, sulfonate anionic surfactants are chosen from those having up to 30 carbon atoms, such as from 6 to 30, from 8 to 28, from 8 to 22 or from 8 to 18 carbon atoms, for example alkyl or alkenyl sulfosuccinates, such as C6-C24 alkyl or alkenyl sulfosuccinates or C8-C18 alkyl or alkenyl sulfosuccinates, alkyl or alkenyl ether sulfosuccinates, such as C6-C24 alkyl or alkenyl ether sulfosuccinates or C8-C18 alkyl or alkenyl ether sulfosuccinates, or acylisethionates, such as such as C6-C24 acylisethionates or C8-C18 acylisethionates. In certain embodiments, the anionic surfactant is chosen from laurylsulfosuccinates.

Carboxylate anionic surfactants comprise at least one carboxylic or carboxylate function (—OOH or —COO⁻) and may optionally also comprise one or more sulfate and/or sulfonate functions. The carboxylic anionic surfactants that may be used thus comprise at least one carboxylic or carboxylate function (—OOH or —COO⁻). They may be chosen from the following compounds: acylglycinates, acyllactylates, acylsarcosinates, acylglutamates, alkyl-D-galactosideuronic acids, alkyl or alkenyl ether carboxylic acids, alkyl(C6-30) or alkenyl aryl ether carboxylic acids, alkylamido or alkenylamido ether carboxylic acids; and also the salts of these compounds; the alkyl, alkenyl and/or acyl groups of these compounds comprising up to 30 carbon atoms, such as from 6 to 30 carbon atoms, especially from 8 to 28, better still from 8 to 22 or even from 8 to 18 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

In certain embodiments, C6-C24 or C8-C18 alkyl or alkenyl monoesters of polyglycosidepolycarboxylic acids, such as C6-C24 or C8-C18 alkyl or alkenyl polyglycoside-citrates, C6-C24 or C8-C18 alkyl polyglycoside-tartrates, C6-C24 or C8-C18 alkyl or alkenyl polyglycoside-sulfosuccinates, and salts thereof, may be chosen.

In further embodiments, polyoxyalkylenated alkyl(amido) or alkenyl(amido) ether carboxylic acids and salts thereof, in particular those comprising from 2 to 50 alkylene oxide and in particular ethylene oxide groups, such as the compounds sold by the company Kao under the name Akypo, may be chosen. For example, polyoxyalkylenated alkyl(amido) or alkenyl(amido) ether carboxylic acids of formula (1) may be chosen:

R₁—(OC₂H₄)ₙ—OCH₂COOA (I)

wherein:
R₁ represents a linear, branched, or cyclic C5-C24 alkyl or alkenyl radical, optionally substituted, an alkyl(C8-C9)phenyl radical, a radical R₂CONH—CH2-CH2- with R₂ denoting a linear or branched C9-C21 alkyl or alkenyl radical, preferably, R₁ is a C8-C20 and preferably C8-C18 alkyl radical, and aryl preferably denotes phenyl;
n is an integer or decimal number (average value) ranging from 2 to 24 and preferably from 2 to 10; and
A denotes H, ammonium, Na, K, Li, Mg, or a monoethanolamine or triethanolamine residue.

In certain embodiments, the polyoxyalkylenated alkyl(amido) or alkenyl(amido) ether carboxylic acids of formula (I) may be those where R₁ is chosen from a C12-C14 alkyl, cocoyl, oleyl, nonylphenyl or octylphenyl radical; A is chosen from a hydrogen or sodium atom, and n ranges from 2 to 20, preferably 2 to 10. In further embodiments, the polyoxyalkylenated alkyl(amido) or alkenyl(amido) ether carboxylic acids of formula (I) may be those where R₁ is chosen from a C12 alkyl radical; A is chosen from a hydrogen or sodium atom, and n ranges from 2 to 10.

In certain embodiments, the carboxylic anionic surfactant may be chosen from acylglutamates, especially of C6-C24 or even C12-C20, such as stearoylglutamates, and in particular disodium stearoylglutamate, acylsarcosinates, especially of C6-C24 or even C12-C20, such as palmitoylsarcosinates, and in particular sodium palmitoylsarcosinate, acyllactylates, especially of C12-C28 or even C14-C24, such as behenoyllactylates, and in particular sodium behenoyllactylate, C6-C24 and especially C12-C20 acylglycinates, (C6-C24)alkyl ether carboxylates and especially (C12-C20)alkyl ether carboxylates, and polyoxyalkylenated (C6-C24)alkyl(amido) ether carboxylic acids, in particular those comprising from 2 to 50 ethylene oxide groups.

As noted herein, the anionic surfactant may optionally be in salt form. In that case, the salt may, for example, be chosen from alkali metal salts, such as the sodium or potassium salt, ammonium salts, amine salts and in particular amino alcohol salts, and alkaline-earth metal salts, such as the magnesium salt. In preferred embodiments, alkali metal or alkaline-earth metal salts may be chosen.

In certain embodiments, the anionic surfactants may be chosen from alkyl or alkenyl ether sulfates, alkyl or alkenyl sulfates, sulfosuccinates, sarcosinates, isethionates, alkyl or alkenyl glucosides, or mixtures thereof. For example, the anionic surfactants may be chosen from sodium laureth sulfate, ammonium capryleth sulfate, ammonium pareth-25 sulfate, ammonium myreth sulfate, ammonium laureth sulfate, sodium decyl ether sulfate, sodium lauryl sulfate, sodium dodecyl sulfate, ammonium lauryl sulfate, disodium laureth sulfosuccinate, diethylhexyl sodium sulfosuccinate, dioctyl sodium sulfosuccinate, cocamidopropyl betaine, sodium lauryl methyl isethionate, sodium N-lauroyl sarcosinate, sodium cocoyl isethionate, disodium laureth sulfosuccinate, sodium cocoamphopropionate, coco glucoside, decyl glucoside, or mixtures thereof. In preferred embodiments, the anionic surfactant is chosen from sodium laureth sulfate, sodium laureth sulfate, optionally containing 2.2 mol of ethylene oxide, sodium lauryl sulfate, sodium lauroyl methyl isethionate, or mixtures containing one or more of these surfactants.

The anionic surfactants may be present in the composition in a total amount up to about 25%, relative to the total weight of the composition. For example, in various embodiments, the anionic surfactants are present in the composition in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%, or may range from about 1% to about 25%, about 1% to about 24%, about 1% to about 23%, about 1% to about 22%, about 1% to about 21%, about 1% to about 20%, about 1% to about 19%, about 1% to about 18%, about 1% to about 17%, about 1% to about 16%, about 1% to about 15%, about 1% to about 14%, about 1% to about 13%, about 1% to about 12%, about 1% to about 11%, about 1% to about 10%, about 1% to about 9%, about 1% to about 8%, about 1% to about 7%, about 1% to about 6%, about 1% to about 5%, about 1% to about 4%, or about 1% to about 3% by weight, relative to the total weight of the composition. In further embodiments, the anionic surfactants may range from about 2% to about 25%, about 2% to about 24%, about 2% to about 23%, about 2% to about 22%, about 2% to about 21%, about 2% to about 20%, about 2% to about 19%, about 2% to about 18%, about 2% to about 17%, about 2% to about 16%, about 2% to about 15%, or about 2% to about 14%, about 2% to about 13%, about 2% to about 12%, about 2% to about 11%, about 2% to about 10%, about 2% to about 9%, about 2% to about 8%, about 2% to about 7%, about 2% to about 6%, about 2% to about 5%, or about 2% to about 4 by weight, relative to the total weight of the composition. In yet further embodiments, the anionic surfactants may range from about 3% to about 25%, about 3% to about 24%, about 3% to about 23%, about 3% to about 22%, about 3% to about 21%, about 3% to about 20%, about 3% to about 19%, about 3% to about 18%, about 3% to about 17%, about 3% to about 16%, about 3% to about 15%, about 3% to about 14%, about 3% to about 13%, about 3% to about 12%, about 3% to about 11%, about 3% to about 10%, about 3% to about 9%, about 3% to about 8%, about 3% to about 7%, about 3% to about 6%, or about 3% to about 5% by weight, relative to the total weight of the composition. In still further embodiments, the anionic surfactants may range from about 4% to about 25%, about 4% to about 24%, about 4% to about 23%, about 4% to about 22%, about 4% to about 21%, about 4% to about 20%, about 4% to about 19%, about 4% to about 18%, about 4% to about 17%, about 4% to about 16%, about 4% to about 15%, about 4% to about 14%, about 4% to about 13%, about 4% to about 12%, about 4% to about 11%, about 4% to about 10%, about 4% to about 9%, about 4% to about 8%, about 4% to about 7%, or about 4% to about 6% by weight, relative to the total weight of the composition. In still further embodiments, the anionic surfactants may range from about 5% to about 25%, about 5% to about 24%, about 5% to about 23%, about 5% to about 22%, about 5% to about 21%, about 5% to about 20%, about 5% to about 19%, about 5% to about 18%, about 5% to about 17%, about 5% to about 16%, about 5% to about 15%, about 5% to about 14%, about 5% to about 13%, about 5% to about 12%, about 5% to about 11%, about 5% to about 10%, about 5% to about 9%, about 5% to about 8%, or about 5% to about 7% by weight, relative to the total weight of the composition.

### Co-Surfactants

The compositions according to the disclosure further comprise at least one co-surfactant. By way of non-limiting example, the co-surfactants may be chosen from short-chain alcohols, e.g. n-alcohols, and short-chain carboxylic acids. For example, the co-surfactant may be chosen from C1-C5 alcohols such as methanol, ethanol, 1-propanol, 1-butanol, or 1-pentanol, or C1-C5 carboxylic acids such as carbonic acid, formic acid, acetic acid, propanoic acid, butanoic acid, acrylic acid, or pentanoic acid. In certain embodiments, the co-surfactant may be chosen from C1-C5 n-alcohols. In further embodiments, the co-surfactant may be chosen from C1-C5 alkyl carboxylic acids.

The co-surfactants may be present in the composition in a total amount up to about 40%, such as up to about 35%, or up to about 30%, relative to the total weight of the composition. For example, in various embodiments, the co-surfactants are present in the composition in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, or may range from about 1% to about 30%, about 2% to about 29%, about 3% to about 28%, about 4% to about 27%, about 5% to about 26%, about 6% to about 25%, about 7% to about 24%, about 8% to about 23%, about 9% to about 22%, about 10% to about 21%, about 10% to about 20%, about 11% to about 19%, about 12% to about 18%, about 13% to about 17%, or about 14% to about 16% by weight, relative to the total weight of the composition. In further embodiments, the co-surfactants may range from about 1% to about 25%, about 2% to about 25%, about 3% to about 25%, about 4% to about 25%, about 5% to about 25%, about 6% to about 25%, about 7% to about 25%, about 8% to about 25%, about 9% to about 25%, about 10% to about 25%, about 11% to about 25%, about 12% to about 25%, about 13% to about 25%, about 14% to about 25%, or about 15% to about 25% by weight, relative to the total weight of the composition. In yet further embodiments, the co-surfactants may range from about 1% to about 20%, about 2% to about 20%, about 3% to about 20%, about 4% to about 20%, about 5% to about 20%, about 6% to about 20%, about 7% to about 20%, about 8% to about 20%, about 9% to about 20%, about 11% to about 20%, about 12% to about 20%, about 13% to about 20%, about 14% to about 20%, about 15% to about 20%, about 16% to about 20%, about 17% to about 20%, about 18% to about 20%, or about 19% to about 20% by weight, relative to the total weight of the composition. In still further embodiments, the co-surfactants may range from about 1% to about 18%, about 2% to about 18%, about 3% to about 18%, about 4% to about 18%, about 5% to about 18%, about 6% to about 18%, about 7% to about 18%, about 8% to about 18%, about 9% to about 18%, about 10% to about 18%, about 11% to about 18%, about 12% to about 18%, about 13% to about 18%, about 14% to about 18%, about 15% to about 18%, about 16% to about 18%, or about 17% to about 18% by weight, relative to the total weight of the composition. In still further embodiments, the co-surfactants may range from about 1% to about 16%, about 2% to about 16%, about 3% to about 16%, about 4% to about 16%, about 5% to about 16%, about 6% to about 16%, about 7% to about 16%, about 8% to about 16%, about 9% to about 16%, about 10% to about 16%, about 11% to about 16%, about 12% to about 16%, about 13% to about 16%, or about 15% to about 16% by weight, relative to the total weight of the composition.

### Cyclic Compounds

In certain embodiments, the compositions according to the disclosure further comprise at least one cyclic compound, although in at least some embodiments, the compositions do not comprise a cyclic compound. By way of non-limiting example, the cyclic compounds may be chosen from aromatic alcohols and cyclic carbonates.

In various exemplary embodiments, the cyclic compounds may be chosen from those of formula (II):

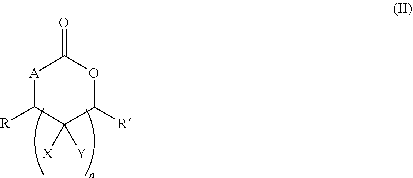

wherein:
A is chosen from N and O;
R and R′ are independently chosen from H, CH₃, CH₂CH₃, and CH₂OH;
X and Y are independently chosen from H and alkyl, for example C1-C6 or C1-C3 alkyl groups, such as CH₃; and
n=0 or 1.

In one embodiment, A is O, in which case formula (II) represents an alkylene carbonate. In one embodiment, R and R′ are both H, and in yet a further embodiment, one of R and R′ is H. In one embodiment, when n is 1, at least one or both X and Y are H.

In an embodiment, n is 0, and formula (II) is a five-membered ring of the following structure:

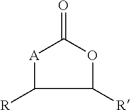

where A, R, and R′ are as defined above.

For example, formula (II) may be chosen from:

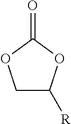

where R is as defined above.

Five-membered alkylene carbonates (1,3-dioxolan-2-ones), such as ethylene carbonate (“EC”, where R and R′═H), propylene carbonate (R═CH3 and R—H), butylene carbonate (where R═CH2CH3 and R—H or where R═CH3 and R—CH3), and glycerol carbonate (R═CH2OH and R—H) may be chosen in certain embodiments.

In further embodiments, six-membered alkylene carbonates (1,3-dioxan-2-ones) may be chosen, such as trimethylene carbonate (X, Y, R, and R′ and H). Further, compounds where A is N such as 2-oxazolidinone (R and R′ are H and n is 0), and derivatives thereof, may be chosen.

In at least certain embodiments, cyclic compounds useful herein may be chosen from small molecules (e.g. MW s 800 g/mol, or <600 g/mol, such as s 200 g/mol) rather than oligomeric compounds or polymers (>1000 g/mol).

For example, the cyclic compound may be chosen from phenoxyalcohols such as 2-phenoxyalcohol, phenoxyethanol, phenylethyl alcohol, and benzyl alcohol, or propylene carbonate, dipropylene carbonate, butylene carbonate, 2,3-butylene carbonate, 2,3-pentylene carbonate, pentylene carbonate, ethylene carbonate, or mixtures thereof.

Without limitation, exemplary cyclic compounds may be chosen from those available from Huntsman Corporation under the tradename JEFFSOL@ alkylene carbonates, such as JEFFSOL EC-75, EC-50, and EC-25 which are blends of JEFFSOL ethylene carbonate and propylene carbonate in the ratios of 75/25, 50/50 and 25/75 by weight, respectively.

The cyclic compounds may be present in the composition in a total amount up to about 30%, such as up to about 25%, or up to about 20%, relative to the total weight of the composition. For example, in various embodiments, the cyclic compounds are present in the composition in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%, or may range from about 1% to about 20%, about 2% to about 19%, about 3% to about 18%, about 4% to about 17%, about 5% to about 16%, about 6% to about 15%, about 7% to about 14%, about 8% to about 13%, about 9% to about 12%, or about 9% to about 11% by weight, relative to the total weight of the composition. In further embodiments, the cyclic compounds may range from about 1% to about 25%, about 2% to about 25%, about 3% to about 25%, about 4% to about 25%, about 5% to about 25%, about 6% to about 25%, about 7% to about 25%, about 8% to about 25%, about 9% to about 25%, about 10% to about 25%, about 11% to about 25%, about 12% to about 25%, about 13% to about 25%, about 14% to about 25%, about 15% to about 25%, about 16% to about 25%, about 17% to about 25%, about 18% to about 25%, about 19% to about 25%, or about 20% to about 25% by weight, relative to the total weight of the composition. In yet further embodiments, the cyclic compounds may range from about 1% to about 20%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 11% to about 20%, about 12% to about 20%, about 13% to about 20%, about 14% to about 20%, about 15% to about 20%, about 16% to about 20%, about 17% to about 20%, or about 18% to about 20% by weight, relative to the total weight of the composition. In still further embodiments, the cyclic compounds may range from about 1% to about 15%, about 2% to about 15%, about 3% to about 15%, about 4% to about 15%, about 5% to about 15%, about 6% to about 15%, about 7% to about 15%, about 8% to about 15%, about 9% to about 15%, about 10% to about 15%, about 11% to about 15%, about 12% to about 15%, or about 13% to about 15% by weight, relative to the total weight of the composition.

### Additional Components

The compositions may comprise additional components, provided they do not substantially interfere with the ability of the composition to remove the direct dyes from the hair fibers but provide conditioning effect to hair fibers. These additional components may be chosen from, for example, anionic polymers, nonionic polymers, rheology modifiers, thickening and/or viscosity modifying agents, associative or non-associative polymeric thickeners, non-polymeric thickeners, non-polymeric cationic surfactants, nacreous agents, dyes or pigments, fragrances, mineral, plant or synthetic oils, waxes, fatty alcohols, lipids, vitamins, proteins including ceramides, vitamins, UV-screening agents, free-radical scavengers, antidandruff agents, hair-loss counteractants, hair restorers, preserving agents, pH stabilizers, solvents, and mixtures thereof.

The additional components may be present in a total amount up to about 90%, such as up to about 80%, up to about 70%, up to about 60%, up to about 50%, or up to about 40% by weight, relative to the total weight of the composition.

Methods of Removing Color from Hair

The compositions according to the disclosure are particularly useful for removing semi-permanent hair color from hair fibers. Without being limited by theory, it is believed that the combination of the at least one anionic surfactant and the at least one co-surfactant, and optionally the at least one cyclic compound, as described herein, act together synergistically to greatly improve the ease and speed of removing direct dyes from the hair fibers.

For example, the compositions described herein may be particularly advantageous for removing azo direct dyes, (poly)methine dyes such as cyanins, hemicyanins and styryls, carbonyl dyes, azine dyes, nitro(hetero)aryl dyes, tri(hetero)arylmethane dyes, porphyrin dyes, phthalocyanin dyes, and natural direct dyes, alone or as mixtures. Without intending to be limited, the following direct dyes are commonly used in semi-permanent hair color, which the compositions according to the disclosure are particularly useful for removing from hair fibers: HC blue 15, hydroxyanthraquinoneaminopropyl methyl morpholinium methosulfate, Basic violet 2, Disperse violet 1, Disperse red 15, Disperse blue 3, Disperse blue 377, Disperse 99, Solvent violet 13, Basic blue 6, HC blue 16, Basic blue 99, HC blue 14, Basic brown 16, Acid green 25, Acid black 1, HC red 7, HC orange 2, 3-Nitro-p-hydroxyethylamino-phenol, Acid red 33, HC violet no. 1, 2-Nitro-5-glyceryl methylaniline, 3-Methylamino-4-nitrophenoxyethanol, 4-Amino-3-nitrophenol, H yellow 9, Acid red 52, Acid orange 7, Acid red 18, HC yellow no. 7, Acid red 92, Acid violet 43, Ext violet 2, Acid green 25, or Acid black 1.

Many direct dyes are cationic direct dyes. Mention may be made of the hydrazono cationic dyes of formulas (Va) and (V′a), the azo cationic dyes (VIa) and (VI′a) and the diazo cationic dyes (VIIa) below:

| | |
|---|---|
| Het⁺—C(R^{a})═N—N(R^{b})—Ar, An⁻ | (Va) |
| Het⁺—N(R^{a})—N═C(R^{b})—Ar, An⁻ | (V′a) |
| Het⁺—N═N—Ar, An⁻ | (VIa) |
| Ar⁺—N═N—Ar″, An⁻ | (VI′a) and |
| Het⁺—N═N—Ar′—N═N—Ar, An⁻ | (VIIIa) |

in which in formulae (Va), (V′a), (VIa), (VI′a) and (VIIa):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₅)alkyl-ammonium such as trimethylammonium;
Ar represents an aryl group, especially phenyl, which is optionally substituted, preferentially with one or more electron-donating groups such as i) optionally substituted (C₁-C₈)alkyl; ii) optionally substituted (C₁-C₈)alkoxy; iii) (di)(C₁-C₈)(alkyl)amino optionally substituted on the alkyl group(s) with a hydroxyl group; iv) aryl(C₁-C₈)alkylamino; v) optionally substituted N—(C₁-C₈)alkyl-N-aryl(C₁-C₈)alkylamino, or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group;
or alternatively the substituent R^{a} with a substituent of Het⁺ and/or R^{b} with a substituent of Ar and/or R^{a} with R^{b} form, together with the atoms that bear them, a (hetero)cycloalkyl;
particularly, R^{a} and R^{b} represent a hydrogen atom or a group (C₁-C₄)alkyl, which is optionally substituted with a hydroxyl group;
An⁻ represents an anionic counterion such as mesylate or halide. In particular, mention may be made of the azo and hydrazono cationic dyes bearing an endocyclic cationic charge of formulae (Va), (V′a) and (VIa) as defined previously. More particularly those of formulae (Va), (V′a) and (VIa) derived from the dyes described in patent applications WO 95/15144, WO 95/01772 and EP-714954, which are incorporated herein by reference in their entirety.

In some cases, the cationic part is derived from those of formulae (V-1) and (VI-1):

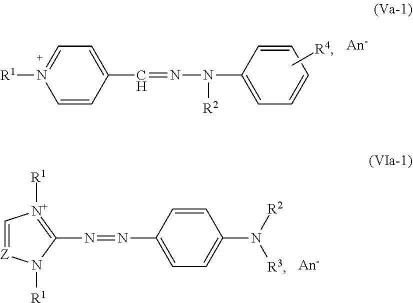

wherein:
R¹ represents a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH; and
An⁻ represents an anionic counterion such as mesylate or halide.

For example, the dye of formulae (Va-1) and (VIa-1) may be chosen from Basic Red 51, Basic Yellow 87 and Basic Orange 31 or derivatives thereof:

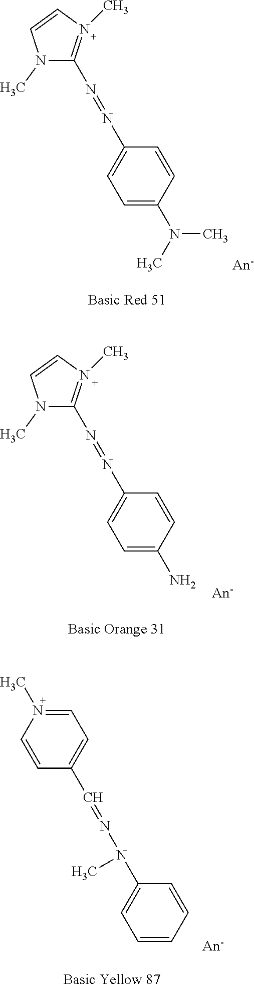

Non-limiting examples of cationic dyes include Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green, Basic Orange 31, 1, Basic Red 2, Basic Red 12 Basic Red 22, Basic Red 76 Basic Red 51, Basic Violet 1, Basic Violet 2, Basic Violet 3, Basic Violet 10, Basic Violet 14, Basic Yellow 57 and Basic Yellow 87.

Non-limiting examples of anionic dyes include Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2, D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium and/or potassium.

Non-limiting examples of nitro dyes include HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Blue 15, HC Blue No. 17, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid.

In the current disclosure, the terms “semi-permanent hair dyes,” “semi-permanent hair color,” and “direct dye” are understood to be used interchangeably without limiting the disclosure.

The methods comprise applying compositions according to the disclosure to hair that has been previously dyed with a semi-permanent hair color, and rinsing the composition from the hair. Optionally, the composition may be left on the hair (“leave-in period”) for a period of time ranging up to about two hours, such as up to about one hour, up to about 45 minutes, up to about 30 minutes, up to about 20 minutes, up to about 15 minutes, or up to about 10 minutes, before the hair is rinsed. For example, the composition may be left on the hair for a period ranging from about 5 to about 120 minutes, such as about 10 to about 60 minutes, or about 15 to about 45 minutes. In one embodiment, the composition may be left on the hair for a period of time ranging from about 20 to about 40 minutes, such as about 20 minutes or about 30 minutes.

Optionally, the hair may be heated after the composition has been applied to the hair and before the hair is rinsed. For example, the hair may be heated at a temperature ranging up to about 100° C., such as from room temperature up to about 80° C., up to about 75° C., up to about 70° C., up to about 65° C., up to about 60° C., up to about 55° C., up to about 50° C., up to about 45° C., or up to about 40° C. In one embodiment, the hair is heated at a temperature ranging from about 25° C. to about 60° C., about 25° C. to about 50° C., about 30° C. to 40° C., about 35° C. to 40° C., or about 38° C. or about 40° C. Heating may be achieved by any means, such as, for example, with a blow dryer or a hood. As used herein, hair that is “heated at a temperature” means the temperature of the heat that the heating means generates. It is to be understood that the methods contemplate that heating may occur for none, some, or all of the period of time the composition is on the hair, without limitation unless specified.

In certain embodiments, the methods comprise multiple applications of compositions described herein to the hair. For example, the methods may comprise applying a composition according to the disclosure to hair fibers, optionally leaving the compositions on the hair for a leave-in period as described herein, optionally with the application of heat as described herein, rinsing the hair fibers, and then applying a second composition according to the disclosure to the hair fibers, optionally leaving the compositions on the hair for a leave-in period as described herein, optionally with the application of heat as described herein, and again rinsing the hair fibers. This cycle may be repeated one, two, three, or more times, as desired, optionally with a period of a few minutes, a few hours, or a few days in between applications. It should be noted that the second, third, etc., composition according to the disclosure applied to the hair fibers may be the same as, or different than, the first, second, etc., compositions applied to the hair fibers, without limitation.

It is to be understood that all definitions herein are provided for the present disclosure only.

It to be understood that, as used herein the terms “the,” “a,” or “an,” mean “at least one,” and should not be limited to “only one” unless explicitly indicated to the contrary. Thus, for example, reference to “a compound” includes examples having two or more such compounds unless the context clearly indicates otherwise.

Unless otherwise expressly stated, it is in no way intended that any method set forth herein be construed as requiring that its steps be performed in a specific order. Accordingly, where a method does not expressly recite an order to be followed by its steps or it is not otherwise specifically stated in the claims or description that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the present disclosure without, however, being limiting in nature.

It will be apparent to those skilled in the art that various modifications and variations can be made in the compositions and methods according to the disclosure without departing from the spirit or scope of the invention. Thus, it is intended that the present invention cover the modifications and variations of this invention provided that they come within the scope of the appended claims and their equivalents.

### EXAMPLES

In the following examples, platinum bleached hair was dyed with Matrix Color Graphics Lacquer (a semi-permanent hair color) which contains 0.2% HC blue 15, a direct dye that is known to be difficult to remove from hair. In each case, after dyeing, the hair was washed six times with DOP shampoo prior to application of the color-removal composition. Next, 1 g of the dyed hair was treated with 40 g of the color-removal composition identified below for 30 minutes at 40° C. The hair was then rinsed with water and blow-dried for evaluation of color removal efficacy.

Color removal in the following examples was evaluated using CIE L* a* b* coordinates. AE is used to describe the color difference, where a greater ΔE value represents more effective removal of color. AE is defined by the following equation:

ΔE=\sqrt{(L*−Lₒ*)²+(a*−aₒ*)²+(b*−bₒ*)²}

where L*, a*, and b* represent the values measured on the dyed hair and L₀*, a₀*, and b₀* represent the values measured on the dyed hair after treatment by the color removal compositions.

Unless otherwise indicated, the ingredient amounts in the compositions/formulations described below are expressed in % by weight, based on the total weight of the composition.

### Baseline Evaluation

For comparison, color removal efficacy of compositions containing the individual anionic surfactants, co-surfactants, and cyclic compounds used in the Examples was determined following the procedure outlined above.

The following color removal compositions were prepared with the balance being water, the hair was treated as disclosed above, and ΔE of the treated hair was calculated.

TABLE A

| Color removal compositions | | ΔE |
|---|---|---|
| 6% | sodium laureth sulfate | 14.4 |
| 15% | ethanol | 2.4 |
| 15% | 1-propanol | 1.6 |
| 15% | 1-butanol | 9.4 |
| 15% | t-butanol | 21.2 |
| 15% | acetic acid | 8.4 |
| 10% | propylene carbonate | 3.6 |
| 10% | butylene carbonate | 8.4 |
| 10% | benzyl alcohol | 9.4 |

### Example 1: Compositions Comprising Anionic Surfactant and Co-Surfactant

Color removal efficacy of compositions containing a combination of anionic surfactants and co-surfactants was determined following the procedure outlined above.

The following color removal compositions were prepared with the balance being water, the hair was treated as disclosed above, and ΔE of the treated hair was calculated.

TABLE 1

| | | | | Expected | |
|---|---|---|---|---|---|
| | | | Individual | Additive | Actual |
| Color removal compositions | | | ΔE | ΔE | ΔE |
| 1-1 | 6% | sodium laureth sulfate | 14.4 | 16.8 | 35 |
| | 15% | ethanol | 2.4 | | |
| 1-2 | 6% | sodium laureth sulfate | 14.4 | 16.0 | 39 |
| | 15% | 1-propanol | 1.6 | | |
| 1-3 | 6% | sodium laureth sulfate | 14.4 | 23.8 | 28.9 |
| | 15% | 1-butanol | 9.4 | | |
| 1-4 | 6% | sodium laureth sulfate | 14.4 | 22.8 | 39.2 |
| | 15% | acetic acid | 8.4 | | |

As can be seen in Table 1, the combination of anionic surfactant and co-surfactant demonstrates significantly greater color removal than either the anionic surfactant or co-surfactant alone. Further, the combination of anionic surfactant and co-surfactant surprisingly synergistically provides a greater than additive color removal effect.

### Example 2: Compositions Comprising Varying Amounts of Anionic Surfactant and Co-Surfactant

Color removal efficacy of compositions containing a combination of anionic surfactants and co-surfactants was determined following the procedure outlined above.

The following color removal compositions were prepared with the balance being water, the hair was treated as disclosed above, and ΔE of the treated hair was calculated.

TABLE 2

| | | | | Expected | |
|---|---|---|---|---|---|
| | | | Individual | Additive | Actual |
| Color removal compositions | | | ΔE | ΔE | ΔE |
| 2-1 | 6% | sodium laureth sulfate | 15.5 | 17.9 | 35.8 |
| | 15% | ethanol | 2.4 | | |
| 2-2 | 6% | sodium laureth sulfate | 15.5 | <17.9 | 24.7 |
| | 7.5% | ethanol | <2.4 | (estimated) | |
| | | | (estimated) | | |
| 2-3 | 10% | sodium laureth sulfate | 16.0 | 18.4 | 37.8 |
| | 15% | ethanol | 2.4 | | |

As can be seen in Table 2, the synergistic combination of anionic surfactant and co-surfactant demonstrates significantly greater color removal than either the anionic surfactant or co-surfactant alone, when varying amounts of anionic surfactant and co-surfactant were chosen. Thus, the synergistic benefit of the combination is expected across the full range of amounts of anionic surfactants and co-surfactants according to the disclosure.

### Example 3: Compositions Comprising Varying Anionic Surfactants and Co-Surfactants

Color removal efficacy of compositions containing a combination of anionic surfactants and co-surfactants was determined following the procedure outlined above.

The following color removal compositions were prepared with the balance being water, the hair was treated as disclosed above, and ΔE of the treated hair was calculated.

TABLE 3

| | | | | Expected | |
|---|---|---|---|---|---|
| | | | Individual | Additive | Actual |
| Color removal compositions | | | ΔE | ΔE | ΔE |
| 3-1 | 10% | poly(ethylene-glyco)-4- | 17.8 | 20.2 | 38.4 |
| | | nonylphenyl-3-sulfopropylether | | | |
| | | potasium salt | | | |
| | 15% | ethanol | 2.4 | | |
| 3-2 | 6% | sodium laureth sulfate | 15.5 | 17.9 | 35.8 |
| | 15% | ethanol | 2.4 | | |
| 3-3 | 6% | sodium lauroyl sarcosinate | * | — | 34.6 |
| | 15% | ethanol | 2.4 | | |
| 3-4 | 6% | sodium cocoyl isethionate | * | — | 32.4 |
| | 15% | ethanol | 2.4 | | |

*not tested

The synergistic combination of anionic surfactant and co-surfactant demonstrates significantly greater color removal than either the anionic surfactant or co-surfactant alone, when alternate anionic surfactants were chosen. Thus, the synergistic benefit of the combination is expected across the full range of anionic surfactants and co-surfactants.

### Example 4: Compositions Comprising Anionic Surfactant, Co-Surfactant, and Cyclic Compound

Color removal efficacy of compositions containing a combination of anionic surfactants, co-surfactants, and cyclic compounds was determined following the procedure outlined above.

The following color removal compositions were prepared with the balance being water, the hair was treated as disclosed above, and ΔE of the treated hair was calculated.

TABLE 4

| | | | | Expected | |
|---|---|---|---|---|---|
| | | | Individual | Additive | Actual |
| Color removal compositions | | | ΔE | ΔE | ΔE |
| 4-1 | 6% | sodium laureth sulfate | 14.4 | 20.4 | 49.4 |
| | 15% | ethanol | 2.4 | | |
| | 10% | propylene carbonate | 3.6 | | |
| 4-2 | 6% | sodium laureth sulfate | 14.4 | 25.2 | 53.2 |
| | 15% | ethanol | 2.4 | | |
| | 10% | butylene carbonate | 8.4 | | |
| 4-3 | 6% | sodium laureth sulfate | 14.4 | 26.2 | 44.7 |
| | 15% | ethanol | 2.4 | | |
| | 10% | benzyl alcohol | 9.4 | | |
| 4-4 | 6% | sodium laureth sulfate | 14.4 | 26.4 | 54.7 |
| | 15% | acetic acid | 8.4 | | |
| | 10% | propylene carbonate | 3.6 | | |
| 4-5 | 6% | sodium laureth sulfate | 14.4 | 32.2 | 49.2 |
| | 15% | acetic acid | 8.4 | | |
| | 10% | benzyl alcohol | 9.4 | | |

As can be seen in Table 4, the combination of anionic surfactant, co-surfactant, and cyclic compound demonstrates significantly greater color removal than any of the anionic surfactant, co-surfactant, or cyclic compound alone. Further, the combination of anionic surfactant, co-surfactant, and cyclic compound surprisingly synergistically provides a greater than additive color removal effect.

### Comparative Examples

Color removal efficacy of comparative compositions containing a combination of anionic surfactants with co-surfactants not within the invention was determined following the procedure outlined above.

The following color removal compositions were prepared with the balance being water, the hair was treated as disclosed above, and ΔE of the treated hair was calculated.

TABLE C

| | | | | Expected | |
|---|---|---|---|---|---|
| Comparative color | | | Individual | Additive | Actual |
| removal compositions | | | ΔE | ΔE | ΔE |
| C-1 | 6% | sodium laureth sulfate | 14.4 | 35.6 | 32.1 |
| | 15% | t-butanol | 21.1 | | |
| C-2 | 6% | sodium laureth sulfate | 14.4 | >14.4 | 14 |
| | 4% | hexanol | * | | |
| C-3 | 6% | sodium laureth sulfate | 14.4 | >14.4 | 12.4 |
| | 15% | sodium octanoate | * | | |

*not tested

As can be seen in Table C, the combination of anionic surfactant with a co-surfactant outside the invention demonstrates less effective color removal than that expected with a composition comprising an anionic surfactant and co-surfactant, in contrast to the inventive compositions.

1. A method for removing direct dye from hair fibers, the method comprising:
applying a composition comprising at least one anionic surfactant and at least one co-surfactant to the hair fibers, and
rinsing the composition from the hair fibers,
wherein the at least one co-surfactant is chosen from C1-C5 alcohols or C1-C5 carboxylic acids.

2. The method of claim 1, wherein the at least one anionic surfactant is chosen from sulfate anionic surfactants, sulfonate anionic surfactants, carboxylate anionic surfactants, or mixtures thereof.

3. The method of claim 2, wherein the sulfate anionic surfactants are chosen from alkyl or alkenyl sulfates, alkyl or alkenyl ether sulfates, alkylamido or alkenylamido ether sulfates, alkylaryl or alkenylaryl polyether sulfates, monoglyceride sulfates, salts thereof, or mixtures thereof.

4. The method of claim 3, wherein the sulfate anionic surfactants are chosen from C6-C30 alkyl or alkenyl sulfates and C6-C30 alkyl or alkenyl ether sulfates optionally having from 2 to 20 ethylene oxide units, salts thereof, or mixtures thereof.

5. The method of claim 2, wherein the sulfonate anionic surfactants are chosen from alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates, alkylsulfolaurates, salts thereof, or mixtures thereof.

6. The method of claim 5, wherein the sulfonate anionic surfactants are chosen from C6-C30 alkylsulfosuccinates, C6-C30 alkyl ether sulfosuccinates, C6-C30 acylisethionates, salts thereof, or mixtures thereof.

7. The method of claim 2, wherein the carboxylate anionic surfactants are chosen from acylglycinates, acyllactylates, acylsarcosinates, acylglutamates; alkyl-D-galactosideuronic acids, alkyl ether carboxylic acids, alkyl(C6-30 aryl) ether carboxylic acids, alkylamido ether carboxylic acids, salts thereof, or mixtures thereof.

8. The method of claim 2, wherein the carboxylate anionic surfactants are chosen from C6-C24 alkyl or alkenyl monoesters of polyglycosidepolycarboxylic acids and polyoxyalkylenated alkyl(amido) ether carboxylic acids of formula (I):
R₁—(OC₂H₄)ₙ—OCH₂COOA (I)
wherein:
R₁ represents a linear, branched, or cyclic C5-C24 alkyl or alkenyl radical, optionally substituted, an alkyl(C8-C9)phenyl radical, a radical R₂CONH—CH2-CH2- with R₂ denoting a linear or branched C9-C21 alkyl or alkenyl radical, preferably, R₁ is a C8-C20 and preferably C8-C18 alkyl radical, and aryl preferably denotes phenyl;
n is an integer ranging from 2 to 24; and
A represents H, ammonium, Na, K, Li, Mg, or a monoethanolamine or triethanolamine residue.

9. The method of claim 2, wherein the at least one anionic surfactant is chosen from sodium laureth sulfate, ammonium capryleth sulfate, ammonium pareth-25 sulfate, ammonium myreth sulfate, ammonium laureth sulfate, sodium decyl ether sulfate, sodium lauryl sulfate, sodium dodecyl sulfate, ammonium lauryl sulfate, disodium laureth sulfosuccinate, diethylhexyl sodium sulfosuccinate, dioctyl sodium sulfosuccinate, cocamidopropyl betaine, sodium lauryl methyl isethionate, sodium N-lauroyl sarcosinate, sodium cocoyl isethionate, disodium laureth sulfosuccinate, sodium cocoamphopropionate, coco glucoside, decyl glucoside, salts thereof, or mixtures thereof.

10. The method of claim 1, wherein the at least one anionic surfactant is present in an amount ranging from about 1% to about 25% by weight, relative to the total weight of the composition, and the at least one co-surfactant is present in an amount ranging from about 5% to about 30% by weight, relative to the total weight of the composition.

11. The method of claim 1, wherein the composition further comprises at least one cyclic compound chosen from aromatic alcohols and cyclic carbonates.

12. The method of claim 11, wherein the at least one cyclic compound is chosen from compounds of formula (II): 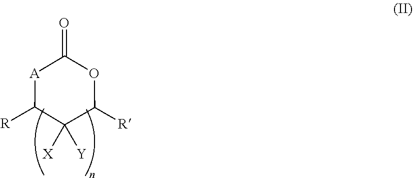
wherein:
A is chosen from N and O;
R and R′ are independently chosen from H, CH₃, CH₂CH₃, and CH₂OH;
X and Y are independently chosen from H and alkyl, for example C1-C6 or C1-C3 alkyl groups, such as CH3; and
n=0 or 1

13. The method of claim 11, wherein the at least one cyclic compound is chosen from 2-phenoxyalcohol, phenoxyethanol, phenylethyl alcohol, benzyl alcohol, propylene carbonate, dipropylene carbonate, butylene carbonate, 2,3-butylene carbonate, 2,3-pentylene carbonate, pentylene carbonate, ethylene carbonate, or a mixture thereof.

14. The method of claim 11, wherein the at least one cyclic compound is present in an amount ranging from about 1% to about 25% by weight, relative to the total weight of the composition.

15. The method of claim 1, wherein the composition is left on the hair for a period of time ranging from about 10 to about 60 minutes.

16. The method of claim 15, wherein the hair is heated at a temperature ranging from about 25° C. to about 60° C.

17. A composition for removing direct dye from hair fibers, the composition comprising:
at least one anionic surfactant chosen from sulfate anionic surfactants, sulfonate anionic surfactants, carboxylate anionic surfactants, or salts thereof;
at least one co-surfactant chosen from C1-C5 alcohols or C1-C5 carboxylic acids; and
at least one cyclic compound chosen from aromatic alcohols or cyclic carbonates.

18. The composition of claim 17, wherein the at least one anionic surfactant is chosen from sodium laureth sulfate, ammonium capryleth sulfate, ammonium pareth-25 sulfate, ammonium myreth sulfate, ammonium laureth sulfate, sodium decyl ether sulfate, sodium lauryl sulfate, sodium dodecyl sulfate, ammonium lauryl sulfate, disodium laureth sulfosuccinate, diethylhexyl sodium sulfosuccinate, dioctyl sodium sulfosuccinate, cocamidopropyl betaine, sodium lauryl methyl isethionate, sodium N-lauroyl sarcosinate, sodium cocoyl isethionate, disodium laureth sulfosuccinate, sodium cocoamphopropionate, coco glucoside, decyl glucoside, salts thereof, or mixtures thereof.

19. The composition of claim 17, wherein the at least one cyclic compound is chosen from 2-phenoxyalcohol, phenoxyethanol, phenylethyl alcohol, benzyl alcohol, propylene carbonate, dipropylene carbonate, butylene carbonate, 2,3-butylene carbonate, 2,3-pentylene carbonate, pentylene carbonate, ethylene carbonate, or a mixture thereof.

20. A composition for removing direct dye from hair fibers, the composition comprising:
at least one anionic surfactant chosen from sodium laureth sulfate, present in an amount ranging from about 1% to about 25% by weight, relative to the total weight of the composition;
at least one co-surfactant chosen from C1-C5 alcohols or C1-C5 alkyl carboxylic acids, present in an amount ranging from about 5% to about 25% by weight, relative to the total weight of the composition; and
at least one cyclic compound chosen from aromatic alcohols or cyclic carbonates, present in an amount ranging from about 1% to about 25% by weight, relative to the total weight of the composition.
